(19) United States
(12) Patent Application Publication
(10) Pub. No.: US 2009/0018641 A1
(43) Pub. Date: Jan. 15, 2009

(54) LARGE VESSEL STENTS

(76) Inventor: Christoph Binkert, Winterthur (CH)

Correspondence Address: FISH & RICHARDSON PC, P.O. BOX 1022, MINNEAPOLIS, MN, 55440-1022, US

(21) Appl. No.: 11/909,537

(22) PCT Filed: Mar. 24, 2006
(86) PCT No.: PCT/US2006/010940

§ 371 Date: Sep. 11, 2008

Related U.S. Application Data
(60) Provisional application No. 60/665,424, filed on Mar. 25, 2005.

(51) Int. Cl. A61F 2/06

(52) U.S. Cl. 623/1.15

(57) ABSTRACT

Large cell stents can be made having a plurality of cylindrical segments; and a plurality of connectors that join the segments to form a hollow tube, in which each segment comprises a series of support elements joined end to end at turning points in a zig-zag pattern to form a cylinder; a first segment is joined to a second segment by a plurality of connectors, each connector connecting a turning point of a first segment to a corresponding turning point of a second segment; and cells of the stent comprise two support elements in a first segment, one connector, two support elements in a second adjacent segment, and a second connector, all connected in series to form a continuous line. In some embodiments, each turning point in the first segment is longitudinally aligned with turning point in the second segment.

### CROSS-REFERENCE TO RELATED APPLICATIONS

This application claims priority to U.S. Application Ser. No. 60/665,424, filed on Mar. 25, 2005, the content of which is hereby incorporated by reference in its entirety.

### TECHNICAL FIELD

This invention relates to intraluminal implants, and more particularly to stents for use in blood vessels.

### BACKGROUND

Stents are widely used for supporting lumen structures in patients' bodies. For example, stents may be used to open occlusion of, or to support, a blood vessel or other body lumen.

Stents are typically tubular structures, and are passed through body lumens in a collapsed state. At the point of an obstruction or other deployment site in the body lumen, the stent is expanded to support the lumen at the deployment site.

Several types of stents are commonly used. Balloon-expandable stents are expanded from a collapsed state to an open state by inflating a balloon within the stent at the deployment site. Another common stent type, a self-expanding stent, can be secured to a stent delivery device under tension in a collapsed state. At the deployment site, the stent is released so that internal tension within the stent causes the stent to self-expand to its expanded diameter. This type of stent is often made of a “super-elastic” material such as a shape-memory alloy. Such shape-memory stents can experience a phase change at the elevated temperature of the human body. The phase change results in expansion from a collapsed state to an expanded state.

### SUMMARY

The invention is based, in part, on the discovery that, by carefully designing a stent with sufficiently large cells, large vessels can be adequately stented while maintaining access to side-branch vessels. These new stents have cells that are large enough to allow a catheter to pass through them. Because of this large cell design, a catheter can pass through a cell of the stent into (or from) the side branch vessel from (or into) the large vessel. Also, the diameter of the stent allows for minimal blood flow impairment to or from the side-branch vessels in the stented area and a decreased risk of thrombosis. The uniform design allows a large vessel to be stented with a stent that is stable, likely to maintain a relatively constant diameter even when bent, and unlikely to collapse or kink. These features lead to increased control and predictability.

In one aspect, the invention features stents that include a plurality of cylindrical segments; and a plurality of connectors that join the segments to form a hollow tube, in which each segment includes a series of support elements joined end to end at turning points in a zig-zag pattern to form a cylinder; a first segment is joined to a second segment by a plurality of connectors, each connector connects a turning point of a first segment to a corresponding turning point of a second segment; and cells of the stent include two support elements in a first segment, a first connector, two support elements in a second adjacent segment, and a second connector, all connected in series to form a continuous line. In some embodiments, each turning point in the first segment is longitudinally aligned with a turning point in the second segment.

In another aspect, the invention features stents that include a plurality of cylindrical segments; and a plurality of connectors that join the segments to form a hollow tube, in which each segment includes a series of support elements joined end to end in a zig-zag pattern to form a cylinder including a series of alternating turning point peaks and nadirs; a first segment is joined to a second segment by a connector extending from each nadir of the first segment to a corresponding peak of the second segment; each peak in the first segment is longitudinally aligned with a peak in the second segment; and cells of the stent are formed of consecutively connected two support elements in a first segment, one connector, two support elements in a second adjacent segment, and a second connector.

In certain embodiments, the stents can have a collapsed state during delivery to a site of implantation and an expanded state once implanted, e.g., the stent can have an expanded diameter of about 12 mm to about 30 mm (e.g., about 15, 18, 20, 23, 25, or 28 mm) and a collapsed diameter of less than about 15 mm (e.g., less than about 13, 12, 11, or 10 mm). The stents can have two to six segments and each segment can be about 1 cm to 2 cm in length. For example, the stent can include two to four segments and six to ten cells circumferentially spaced apart along the longitudinal axis of the stent.

The cell of the stent can have a sufficient size for a catheter with a 14.5F diameter to pass through the cell. In various embodiments, the catheter is able to pass through a cell that is compressed or enlarged.

In some embodiments, the connector has a length that is approximately one half of a distance between two adjacent turning points in a segment. In some embodiments, the connector has a length that is approximately one half of the width between two peaks or nadirs in a segment. In various embodiments, the stent is relatively inflexible, and the stents include an alloy, e.g., a shape-memory alloy such as nitinol. The term “relatively inflexible” refers to a stent that does not bend more than about 30° per segment, measured from a longitudinal central axis.

In other embodiments, the stents include a biodegradable polymer, a bioerodable polymer, or a bioresorbable material. The term “biodegradable” means a material that is broken down into components smaller than its original size when present in a target system, e.g., a living system. The term “bioresorbable” means a material that is capable of being absorbed by, and integrated into, a system, e.g., a living system, when placed into the system or when created and subsequently placed in the system. The term “bioerodable” means that after administration, a material is degraded in vivo, through enzymatic action and/or as a consequence of non-enzymatic hydrolysis, into non-toxic products that are subject to catabolism, metabolism, or excretion.

In certain embodiments, the stents can include a coating, e.g., a radiopaque material (e.g., coating) or a coating that includes (and optionally releases) a drug. The stent can be configured to fit into a vessel about 12 mm to about 30 mm in diameter, e.g., aorta, iliac arteries, brachiocephalic trunk, inferior vena cava, superior vena cava, brachiocephalic veins, and iliac veins.

In certain embodiments, the stent is cut from a hollow tube of material such that all support elements and connectors are made from one continuous piece of material. In other embodiments, the stent is made from one continuous wire of material bent and connected to form the individual support elements and connectors.

The new stents can be expandable between a collapsed state and an expanded state; and can include a plurality of cylindrical segments; and a plurality of connectors that join the segments to form a hollow tube, in which each segment includes a series of support elements joined end to end at turning points in a zig-zag pattern to form a cylinder; a first segment is joined to a second segment by a plurality of connectors, each connector connects a turning point of a first segment to a corresponding turning point of a second segment; and cells of the stent include two support elements in a first segment, a first connector, two support elements in a second adjacent segment, and a second connector, all connected in series to form a continuous line.

In another aspect, the invention relates to methods of stenting a large vessel (e.g., aorta, iliac arteries, brachiocephalic trunk, inferior vena cava, superior vena cava, brachiocephalic veins, and iliac veins). The methods include obtaining a stent as described herein; placing the stent at a deployment site; and enabling the stent to attain an expanded state. In certain embodiments, a side-branch vessel is overstented and a catheter is passed through a cell of the stent into (or from) an overstented side-branch vessel from (or into) the stented large vessel. In various embodiments, the stenting produces minimal blood flow impairment in the side-branch vessel or in the large vessel and can result in a decreased risk of thrombosis. The new methods can also include delivering the stent in a collapsed state (e.g., on a balloon catheter) to the deployment site; and expanding a balloon within the stent to expand the stent. The methods also include delivering the stent in a collapsed state (e.g., the stent is a self-expanding stent and the stent is positioned on a delivery catheter and held in the collapsed state by a retractable sheath) to the deployment site; and retracting the sheath to enable the stent to expand.

For example, the new methods can be used to treat May-Thurner Syndrome, permit a central venous catheter to be passed through a stent placed in the superior vena cava, and can be used to place the stent in the brachiocephalic trunk and accessing the right subclavian and common carotid arteries.

In another aspect, the invention relates to methods of making the new stents by obtaining a sheet of stent material; forming a tube of the stent material of a desired size; and cutting (e.g., precision cutting or laser cutting) the stent from the tube of stent material. The methods can include shaping the cut sheet into a shape compatible for use as a stent and can also include fixing the stent in the shape compatible for use as a stent. The stent material can include an alloy (e.g., nitinol) or stainless steel.

In another aspect, the invention relates to methods of making a new stent by obtaining a single continuous strand of stent material; and forming (e.g., weaving) the stent out of the single strand of stent material. The stent material can be an alloy, stainless steel, a biodegradable polymer, a bioerodable polymer, a dissolvable polymer, or a bioresorbable material.

Unless otherwise defined, all technical and scientific terms used herein have the same meaning as commonly understood by one of ordinary skill in the art to which this invention belongs. Although methods and materials similar or equivalent to those described herein can be used in the practicing or testing of the present invention, suitable materials and methods are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features, objects, and advantages of the invention will be apparent from the description and drawings, and from the claims.

### DESCRIPTION OF DRAWINGS

FIG. 1 is a three-dimensional view of a stent when the stent is assembled as a straight tube. A cell of the stent is shaded.

FIGS. 2A and 2B are three-dimensional views of two segments of the stent in FIG. 1.

FIG. 2A is a view of the stent laid straight and FIG. 2B is a view of the stent following bending of the stent around a curve. The cells of the stent in FIG. 2B are compressed on the inner surface of curvature and enlarged on the outer surface of curvature.

FIG. 3 is a plan view of the peripheral surface of a stent of the disclosure as it would appear if longitudinally split and laid out flat and straight. Two segments are shown.

FIGS. 4A and 4B are plan views of the peripheral surface of the stent in FIG. 3 following bending of the stent. FIG. 4A is a view of the compressed cells on the inner surface of curvature of the stent and FIG. 4B is a view of the enlarged cells on the outer surface of curvature of the stent.

Like reference symbols in the various drawings indicate like elements.

### DETAILED DESCRIPTION

The invention features stents that can be used in large vessels and that allow for access into (or from) side-branched vessels from (or into) the stented large vessel.

Stent Design

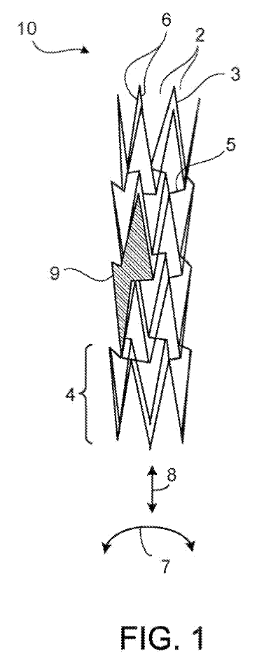

The stent is a reticulated hollow tube. The dimensions of the stent allow its use in large vessels. FIG. 1 is a three-dimensional view of the stent when the stent 10 is laid straight. The diameter of the stent can be about 12 mm to about 30 mm; for example, the stent can be 12, 15, 18, 21, 24, 27, or 30 mm in diameter. The stent 10 is made up of segments 4 and can have two, three, four, five, six, or more segments 4. A segment 4 is a section of the stent 10 that extends annularly and is made up of annular support portions 2. Each annular support portion 2 is a V-shaped component made up of two support elements 3. The support elements 3 are arranged in a zig-zag pattern and connected to each other at turning points 6. The turning points can be peaks or nadirs. Each segment 4 is about 1 cm to 2 cm, e.g., 1.5 cm, in length. A cell 9 of the stent 10 is shaded. A cell 9 is an open structure of the stent 10 and is the opening created by two annular support portions 2, where the annular support portions 2 are in adjacent segments 4 in which the turning points 6 of each annular support portion 2 point away from the other turning point 6, and the annular support portions 2 are connected to two connectors 5, where each connector 5 is attached to two turning points 6. The length of the connectors 5 is approximately one half of the width of the widest part of an annular support portion 2. Each cell 9 is large enough to allow a catheter with a 14.5F diameter to pass through. The catheter is able to pass through a cell 9 that is compressed, for example, on the inner surface of a bent stent; and able to pass through a cell 9 that is enlarged, for example, on the outer surface of a curved stent. In this embodiment, the stent 10 is made up of four segments 4 and six cells 9 make up the circumference of the stent 10. The cells 9 are circumferentially spaced apart along the longitudinal axis 8 of the stent 10.

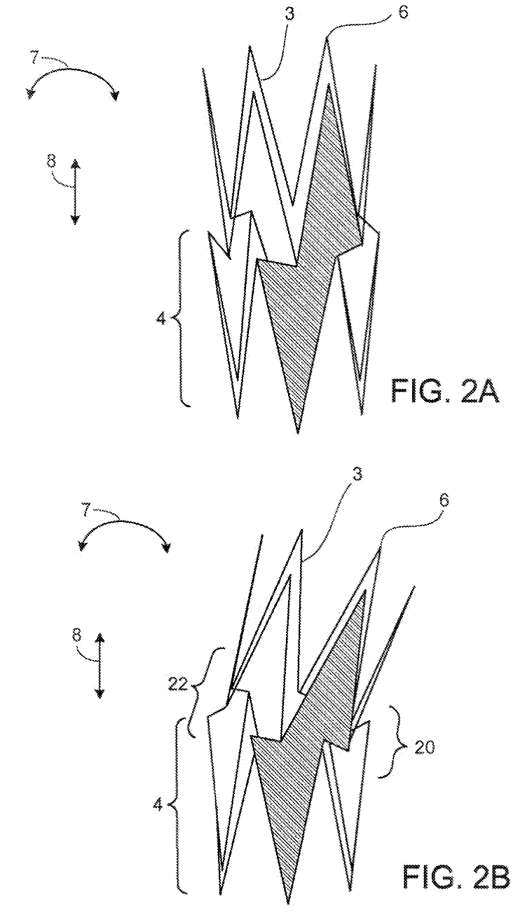

FIGS. 2A and 2B are three-dimensional views of two segments 4 of the stent 10 in FIG. 1. FIG. 2A is a view of the two segments 4 laid straight. FIG. 2B is a view of the two segments 4 following bending of the stent 10 around a curve. In FIG. 2B, the cells 9 of the stent 10 are compressed on the inner surface of curvature 20 and enlarged on the outer surface of curvature 22.

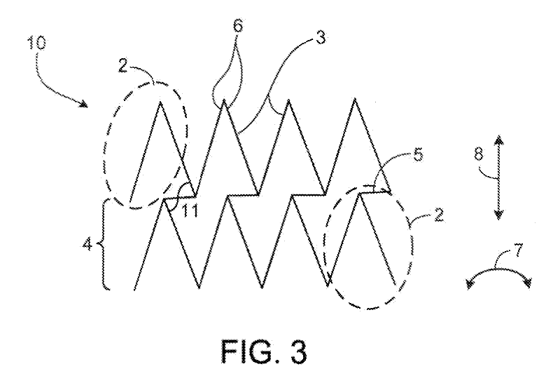

FIG. 3 is a plan view of the peripheral surface of an embodiment of a stent 10, e.g., the stent shown in FIG. 2A, as it would appear if longitudinally cut and laid out flat and straight, with a number of annular support portions 2. In this embodiment, the stent consists of two segments 4 made up of annular support portions 2, each of which is connected between segments 4 along the longitudinal axis 8 of the stent 10 by connectors 5 which extend in a direction perpendicular to the longitudinal axis 8 of the stent 10. Two support elements 3 are connected at a turning point 6 and are arranged in a V-shape. Within a segment, the turning points 6 alternate to point in opposite directions along the longitudinal axis 8 of the stent 10, i.e., the turning points 6 make alternating peaks and nadirs. The segments 4 can extend curvedly in a first direction indicated by the double-headed arrow 7, along the longitudinal axis 8 of the stent 10. Four support elements 3 of two annular support portions 2, where the annular support portions are in adjacent segments 4, and two connectors 5 create a cell 9 of the stent 10 and each connector 5 is attached to two turning points 6. The angle of the connection of a connector 5 with a support element 3 forms an acute angle 11. In the illustrated embodiment, the support elements 3 extend in a zig-zag pattern in the direction perpendicular to the longitudinal axis 8 of the stent 10. As a result, the manufacture of the stent 10 can be particularly simple, by virtue of the simple geometry involved, with a desirable distribution of stresses over the annular support elements 3.

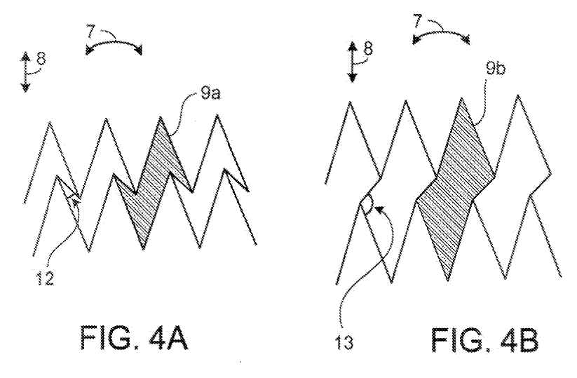

FIGS. 4A and 4B are plan views of the stent shown in FIG. 2A when the stent is curved relative to the longitudinal axis 8 as shown in FIG. 2B. If, for example, a force is applied to the stent 10 in direction of arrow 7, FIG. 4A shows that on the inner surface of curvature (i.e., the side of the stent 10 which faces towards the center point of the curvature, labeled 20 in FIG. 2B), the connectors 5 are aligned to be more in line with the longitudinal axis 8 of the stent 10 and less perpendicular relative to the longitudinal axis 8 of the stent 10 and such that the connectors 5 form a more acute angle 12 (relative to angle 11) with the support elements 3 in the direction perpendicular to the longitudinal axis 8 of the stent 10, with the result that the area of the cell 9a of the stent 10 is reduced relative to the area of the cell 9 in FIG. 3. Also, the length of the cell 9a is reduced along the longitudinal axis 8 of the stent 10 relative to the length of the cell 9 in FIG. 3. This is a “compressed” configuration or “compressed state.”

FIG. 4B shows that on the outer surface of curvature (i.e., the side of the stent 10 which faces away from the center point of curvature, labeled 22 in FIG. 2B), the connectors 5 are aligned to be more in line with the longitudinal axis 8 of the stent and less perpendicular relative to the longitudinal axis 8 of the stent 10 and such that the connectors 5 form a less acute angle 13 (relative to angle 11) with the support elements 3 in the direction perpendicular to the longitudinal axis 8 of the stent 10, with the result that the area of the cell 9b of the stent 10 is enlarged relative to the area of a cell 9 in FIG. 3. Also, the length of the cell 9b is increased along the longitudinal axis 8 of the stent 10 relative to the length of the cell 9 in FIG. 3. This is an “enlarged” configuration or “enlarged state.”

The entire surface of the stent 10 can be collapsed relatively uniformly for delivery to a deployment site. In this collapsed state, the diameter of the stent 10 is less than, e.g., approximately half, the diameter of the stent 10 in an expanded state. Upon delivery to the deployment site, the stent is either actively expanded to the expanded state, i.e., the diameter is increased relative to the diameter of the stent 10 in a collapsed state, or automatically expands upon removal from the delivery sheath or catheter.

The uniform design of the stent increases the stability of the stent and allows the stent to maintain a relatively constant diameter, even when bent. In contrast, stents that are made up of segments sutured together can kink and collapse at the suture positions. As a result, such sutured stents can be unpredictable and the diameter of such stents may not be constant, i.e., the diameter of such stents at the suture positions may be significantly reduced as compared to the diameter over other areas of the stent, especially when the stent is bent. The design of the present stent allows the stent to maintain a relatively constant diameter even when bent, and the stent is less likely to collapse or kink.

The stent has limited flexibility. Because of the relatively long segment length, the stent is less flexible than other stents. It is estimated that, in many cases, the stent should not be bent more than about 30° (e.g., not more than about 25°, 20°, 15°, 10° or 5°) per segment in order to keep a constant diameter throughout the stented vessel segment. However, if needed, the stent may be bent more; this can be determined by the physician's judgment.

The stent can be made of a variety of materials such as gold; titanium; platinum; tantalum; alloys such as shape-memory alloys, e.g., nickel-titanium-based alloys, e.g., nitinol, cobalt-chromium-based alloys, tantalum-based alloys, cobalt-chromium-nickel-based alloys, e.g., CONICHROME®, PHYNOX™, ELGILOY®, and MP35N®, titanium-based alloys, titanium-zirconium-niobium-based alloys, titanium-aluminum-vanadium-based alloys, e.g., TI-6A1-4V; stainless steel; biodegradable polymers and bioresorbable materials such as polyesters, polyorthoesters, polyanhydrides, poly(imidocarbonate)s, poly(phosphazene)s, cyclic phosphate monomers, polylactide and trimethylene carbonate blends, poly(L-lactic acid), poly(D,L-lactic acid), polycaprolactone, poly(glycolic acid), chitosan, sulfonated chitosan, or natural polymers or polypeptides, e.g., reconstituted collagen, spider silk, polyamides, polyurethanes, polylactides, polyglycolides, polydioxanones, poly(lactide-co-glycolide), poly(glycolide-co-polydioxanone), poly(glycolide-co-trimethylene carbonate), poly(glycolide-co-caprolactone), other caprolactone derivatives, poly(ethylene terephthalate), poly(butyric acid), poly(valeric acid), poly(lactide-co-caprolactone), and blends and copolymers thereof; and bioerodable polymers (U.S. Pat. Nos. 6,709,455; 6,805,876; 6,858,222; 6,863,684; U.S. Pat. App. No. 2005/0058603). An example of a bioresorbable material that has no reported negative effects is described in Pat. App. No. 2004/0098108; Blindt et al. provides an example of bioresorbable polyesters like poly(D,L-lactide) ((1999) Int. J. Artif. Organs 22:843-53); see also U.S. Pat. App. No. 2005/0048121.

In addition, the stent can be coated with a material that releases a drug, such as an immunosuppressant or combination of immunosuppressants, e.g., mycophenolic acid, rapamycin, mizoribine, riboflavin, tiazofurin, methylprednisolone, FK 506, zafurin, cyclosporine, or methotrexate, alone or in combination with another substance with which the stent can be coated, e.g., an anti-platelet agent, an anti-thrombotic agent, or IIb/IIIa agent (U.S. Pat. Nos. 6,858,221 and 6,641,611). The stent can be coated with a radiopaque coating such as platinum, gold, tungsten, or tantalum. The stent can also be coated with a biocompatible material, such as parylene or polyethylene glycols, in the event that the stent is made of a material that is not biocompatible.

For example, the stent can be made of a super-elastic e.g., shape-memory, alloy. Such a material has the property that when deformed and heated past a critical temperature, it “remembers” its deformed shape. When cooled and subjected to further deformation, such a stent springs back to this remembered shape. A suitable super-elastic metal from which the stent can be manufactured is a nickel-titanium alloy, e.g., nitinol. In the case of a nickel-titanium alloy, the critical temperature is approximately 700 degrees Fahrenheit. An attractive feature of a material such as nitinol is that it is NMR compatible.

The stent can be made by cutting the stent from a tube of the stent material. For example, when forming the stent from a shape-memory alloy such as nitinol, the stent can be laser cut from a nitinol tube. Thereafter, the stent can be subjected to a shape-setting process in which the cut tube is expanded on a mandrel and then heated. Multiple expansion and heating cycles can be used to shape-set the stent to the final expanded diameter.

Other methods may also be used to make the stent. The stent can be made by precision cutting, e.g., laser cutting, chemical etching, water jet cutting, or standard tool machining a tube or sheet of the stent material. If a sheet is used, the sheet is shaped into a shape compatible for use as a stent, e.g., a tubular structure, and may optionally be secured in that shape, e.g., fused or welded. As other alternatives, the stent may be woven, braided, knit, or made by some combination of these methods out of strands of the stent material.

Uses

In use, the stent is advanced to a deployment site in a lumen such as a blood vessel (e.g., an occlusion site in need of circumferential support) while in the collapsed state, i.e., having a reduced diameter. The stent is then expanded at the deployment site. The stent may be expanded through any conventional means, for example, the stent may be balloon-expandable or self-expanding. For a balloon-expandable stent, the stent in the reduced diameter may be placed at the tip of a balloon catheter. At the deployment site, the stent is expanded (e.g., through expansion of the balloon), thereby causing the stent to expand from a collapsed state to an expanded state, i.e., having an expanded diameter. A preferred material for balloon-expandable stents is stainless steel. For self-expanding stents, the stent may be formed of a shape memory alloy, such as nitinol. To position the self-expanding stent at a deployment site, the stent can be mounted on a delivery catheter. As is conventionally known in the art, the stent can be held in a collapsed state in the delivery catheter by a retractable sheath. As is also known in the art, the delivery catheter can be used to advance the stent to the deployment site (e.g., a constricted region of a vessel). At the deployment site, the sheath is retracted, thereby releasing the stent. Once released, the stent self-expands to the expanded state.

The stent can be used in vessels such as arteries, e.g., aorta (thoracic and abdominal), iliac arteries, brachiocephalic trunk (also known as the innominate artery), and veins, e.g., inferior and superior vena cava (“IVC” and “SVC,” respectively), brachiocephalic veins (also known as innominate veins), iliac veins (especially the left common iliac vein). The vessels are about 12 mm to about 30 mm in diameter.

The stent can be used to open occlusions in large, i.e., main, vessels or to prevent recoil or restenosis after angioplasty. The large cell design of the stent allows overstenting of a side-branch vessel while maintaining access to the side-branch vessel from the main vessel and to the main vessel from the side-branch vessel. For example, the large cell design allows a catheter to pass through a cell of the stent positioned in a main vessel into or from the side-branched vessel. This feature is important, for example, for placing a central venous catheter in the SVC and for stenting of the brachiocephalic trunk while maintaining access to the overstented right subclavian and common carotid arteries. Examples of catheters include catheters with a 14.5F diameter, central venous catheters, and dialysis catheters or a central vein access for paraenteral nutrition, chemotherapy and other agents which have to be delivered into the central veins.

The large cell design allows overstenting of side-branch vessels with minimal blood flow impairment to or from the side-branch vessel, which can result in a decreased risk of thrombosis. Overstenting a side-branch vessel is a concern because of the risk of thrombosis of the side-branch vessel. However, quite often narrowings of blood vessels are located at the site of side-branch vessels because of flow changes at that area. Therefore, overstenting of one or more side-branch vessels cannot be avoided. If less material impairs the flow to or from the side-branch vessel, the risk for thrombosis decreases. The new stent design has a network of large cells and minimizes flow reduction through the stent cells. For example, this feature is useful in treating conditions such as May-Thurner syndrome in which the obstruction is in the central portion of the left common iliac vein. Stenting from the left common iliac vein to the IVC is necessary and covers the inflow of the right common iliac vein. If less material is covering the right iliac vein inflow, the risk of a deep vein thrombosis decreases.

Intimal hyperplasia, a reaction of a vessel against a stent, can create a risk of blood flow impairment by decreasing the diameter of a vessel by up to 2 or 3 mm. The stent of the present disclosure has a large diameter and can be used in large vessels; therefore, only minimal effects on blood flow through the stented vessel occur because the amount by which the diameter decreases due to hyperplasia is small relative to the diameter of the stent.

### OTHER EMBODIMENTS

It is to be understood that while the invention has been described in conjunction with the detailed description thereof, the foregoing description is intended to illustrate and not limit the scope of the appended claims. Other aspects, advantages, and modifications are within the scope of the following claims.

1. A stent comprising
a plurality of cylindrical segments; and
a plurality of connectors that join the segments to form a hollow tube,
wherein
each segment comprises a series of support elements joined end to end at turning points in a zig-zag pattern to form a cylinder;
a first segment is joined to a second segment by a plurality of connectors, each connector connecting a turning point of a first segment to a corresponding turning point of a second segment; and
cells of the stent comprise two support elements in a first segment, one connector, two support elements in a second adjacent segment, and a second connector, all connected in series to form a continuous line.

2. The stent of claim 1, wherein each turning point in the first segment is longitudinally aligned with a turning point in the second segment.

3. The stent of claim 1, wherein the stent comprises a collapsed state during delivery to a site of implantation and an expanded state once implanted.

4. The stent of claim 3, wherein the stent has an expanded diameter of about 12 mm to about 30 mm and a collapsed diameter of less than about 15 mm.

5. The stent of claim 3, wherein the stent has an expanded diameter of about 20 mm and a collapsed diameter of less than about 10 mm.

6. The stent of claim 1, wherein the stent comprises two to six segments.

7. The stent of claim 1, wherein each segment is about 1 cm to 2 cm in length.

8. The stent of claim 1, wherein the cell of the stent has a sufficient size for a catheter with a 14.5F diameter to pass through the cell.

9. The stent of claim 8, wherein the catheter is able to pass through a cell that is compressed.

10. The stent of claim 8, wherein the catheter is able to pass through a cell that is enlarged.

11. The stent of claim 1, wherein the connector has a length that is approximately one half of a distance between two adjacent turning points in a segment.

12. The stent of claim 1, wherein the stent comprises two to four segments and six to ten cells circumferentially spaced apart along the longitudinal axis of the stent.

13. The stent of claim 1, wherein the stent is relatively inflexible.

14. The stent of claim 1, wherein the stent comprises an alloy.

15. The stent of claim 14, wherein the alloy comprises a shape-memory alloy.

16. The stent of claim 15, wherein the shape-memory alloy comprises nitinol.

17. The stent of claim 1, wherein the stent comprises a biodegradable polymer, a bioerodable polymer, or a bioresorbable material.

18. The stent of claim 1, wherein the stent comprises a coating.

19. The stent of 18, wherein the coating comprises a radiopaque material.

20. The stent of claim 18, wherein the coating comprises a drug.

21. The stent of claim 1, wherein the stent is cut from a hollow tube of material such that all support elements and connectors are made from one continuous piece of material.

22. The stent of claim 1, wherein the stent is made from one continuous wire of material bent and connected to form the individual support elements and connectors.

23. A method of stenting a large vessel, the method comprising:
obtaining a stent of claim 1;
placing the stent at a deployment site; and
enabling the stent to attain an expanded state.

24. The method of claim 23, wherein the large vessel is selected from the group consisting of: aorta, iliac arteries, brachiocephalic trunk, inferior vena cava, superior vena cava, brachiocephalic veins, and iliac veins.

25. The method of claim 23, wherein a side-branch vessel is overstented and the method further comprises passing a catheter through a cell of the stent into an overstented side-branch vessel from the stented large vessel.

26. The method of claim 23, wherein placing the stent at a deployment site comprises inserting the stent in the superior vena cava and the method further comprises passing a central venous catheter though the stent.

27. The method of claim 23, wherein the stent is placed in the brachiocephalic trunk and the method further comprises accessing the right subclavian and common carotid arteries.

28. The method of claim 23, further comprising:
delivering the stent in a collapsed state to the deployment site, wherein the stent in the collapsed state is delivered on a balloon catheter; and
expanding a balloon within the stent to expand the stent.

29. The method of claim 23, further comprising:
delivering the stent in a collapsed state to the deployment site, wherein the stent is a self-expanding stent and the stent is positioned on a delivery catheter and held in the collapsed state by a retractable sheath; and
retracting the sheath to enable the stent to expand.

30. A method of making a stent, the method comprising:
obtaining a sheet of stent material;
forming a tube of the stent material of a desired size; and
cutting the stent from the tube of stent material, wherein the stent comprises
a plurality of cylindrical segments: and
a plurality of connectors that join the segments to form a hollow tube,
wherein
each segment comprises a series of support elements joined end to end at turning points in a zigzag pattern to form a cylinder;
a first segment is joined to a second segment by a plurality of connectors, each connector connecting a turning point of a first segment to a corresponding turning point of a second segment; and
cells of the stent comprise two support elements in a first segment, one connector, two support elements in a second adjacent segment, and a second connector, all connected in series to form a continuous line.

31. The method of claim 30, wherein the cutting is laser cutting.

32. The method of claim 31, further comprising shaping the cut sheet into a shape compatible for use as a stent.

33. The method of claim 32, further comprising fixing the stent in the shape compatible for use as a stent.

34. The method of claim 30, wherein the stent material comprises an alloy or stainless steel.

35. The method of claim 34, wherein the stent material comprises nitinol.

36. A method of making a stent, the method comprising:
obtaining a single continuous strand of stent material; and
forming the stent out of the single strand of stent material, wherein the stent comprises
a plurality of cylindrical segments; and
a plurality of connectors that join the segments to form a hollow tube,
wherein
each segment comprises a series of support elements joined end to end at turning points in a zigzag pattern to form a cylinder;
a first segment is joined to a second segment by a plurality of connectors, each connector connecting a turning point of a first segment to a corresponding turning point of a second segment; and
cells of the stent comprise two support elements in a first segment, one connector, two support elements in a second adjacent segment and a second connector, all connected in series to form a continuous line.

37. The method of claim 36, wherein the stent material is selected from the group consisting of an alloy, stainless steel, a biodegradable polymer, a bioerodable polymer, a dissolvable polymer, and a bioresorbable material.
